(19) United States
(12) Patent Application Publication
(10) Pub. No.: US 2014/0155321 A1
(43) Pub. Date: Jun. 5, 2014

(54) Polypeptides and Their Use

(71) Applicants: Deborah O'NEIL, Craibstone (GB); Derry MERCER, Craibstone (GB); Colin STEWART, Craibstone (GB)

(72) Inventors: Deborah O'NEIL, Craibstone (GB); Derry MERCER, Craibstone (GB); Colin STEWART, Craibstone (GB)

(73) Assignee: Novabiotics Limited, Craibstone (GB)

(21) Appl. No.: 14/004,344

(22) PCT Filed: Mar. 14, 2013
(86) PCT No.: PCT/GB2013/000112

§ 371 Date: Sep. 10, 2013

Related U.S. Application Data
(60) Provisional application No. 61/620,833, filed on Apr. 5, 2012.

(30) Foreign Application Priority Data
Mar. 14, 2012 (GB) 1204457.4

(51) Int. Cl. A61K 38/16

(52) U.S. Cl. CPC A61K 38/16
USPC 514/3.3; 530/324

(57) ABSTRACT

The present invention relates to a polypeptide or a product comprising said polypeptide for use in the treatment and/or prevention of a fungal infection caused by Malassezia spp. and/or a Malassezia spp associated condition wherein the polypeptide comprises a sequence of about 25 to 200 amino acids wherein substantially all of the amino acids in said sequence are lysine; pharmaceutical compositions comprising said polypeptide or product and uses thereof.

### FIELD OF THE INVENTION

This invention relates to polypeptides and their use in the treatment of fungal infections caused by Malassezia spp.

### BACKGROUND TO THE INVENTION

There remain very few options for the effective treatment of all forms of seborrhoeic dermatitis caused by Malassezia spp., because of a lack of effective active agents that kill the causative organism rather than inhibit its growth and that can be used frequently, and as such have an appropriate safety profile for something used as a consumer health product.

The treatment options for infections contributed to or caused by Malassezia spp. are severely limited and there is a need to discover new therapies which kill such organisms.

### STATEMENTS OF THE INVENTION

The present invention is based in part on the finding that polypeptides of between 25 and 200 lysine residues are highly fungicidal against Malassezia spp. whilst at the same time avoiding certain toxicity issues associated with certain other polylysine polypeptides and as such are effective in the treatment of Malassezia spp. infections in particular topical infections.

According to a first aspect the invention provides a polypeptide for use in the treatment and/or prevention of a fungal infection caused by Malassezia spp. and/or a Malassezia spp associated condition wherein the polypeptide comprises a sequence of 25 to 200 amino acids wherein substantially all of the amino acids in said sequence are lysine.

Polypeptides according to the invention have advantages over respective polypeptides of more than 200 amino acid residues since they do not have associated synthesis and cell toxicity issues. Moreover, polypeptides according to the invention have advantages over respective polypeptides of fewer than 25 amino acid residues since they have improved efficacy against Malassezia spp.

As used herein “substantially” is a relative modifier intended to indicate permissible variation from the characteristic so modified. Specifically, by “substantially all of the amino acids in said sequence of 25 to 200 amino acids are lysine” it is meant that either all, or a high proportion of, the amino acids in the sequence are lysine. By “high proportion”, it is contemplated that 1 or 2 non-lysine, for example glycine, histidine or arginine, substitutions may be made in the sequence.

Preferably the polypeptide comprises a sequence of 25 to 200 consecutive lysine residues. In one embodiment, the polypeptide consists of a sequence of 25 to 200 consecutive lysine residues.

Preferably the polypeptide of the invention is polylysine, for example poly-L-lysine.

In a preferred aspect the polypeptide of the invention comprises a sequence of about 38 to 189 amino acids, including 38 to 161, for example 77 to 155, amino acids wherein substantially all of the amino acids in said sequence of amino acids are lysine. Preferably still, the polypeptide of the invention comprises a sequence of about 50 to 150, for example 50 to 125, including 50 to 75, amino acids wherein substantially all of the amino acids in said sequence are lysine.

The invention also includes known isomers (structural, stereo-, conformational & configurational) and structural analogues of the above amino acids, including peptidomimetics, and those modified either naturally (e.g. post-translational modification) or chemically, including, but not exclusively, phosphorylation, glycosylation, sulfonylation and/or hydroxylation.

In addition, the amino acid sequence of the polypeptide can be modified so as to result in a polypeptide variant that includes the substitution of at least one (for example one or two) amino acid residues in the polypeptide for another amino acid residue including substitutions that utilise the D rather than L form, wherein the variant retains some (typically at least 10%) or all of the biological activity of the corresponding non-variant polypeptide. Thus, the invention provides a polypeptide variant in which one or more lysine is substituted by one or more residues other residues, for example arginine or histidine.

The term “polypeptide” as used herein means, in general terms, a plurality of amino acid residues joined together by peptide bonds. It is used interchangeably and means the same as protein.

The polypeptides of the invention generally are synthetic polypeptides. The polypeptides may be isolated, purified polypeptides or variants thereof, which can be synthesised in vitro, for example, by a solid phase polypeptide synthetic method, by enzyme-catalysed polypeptide synthesis or with the aid of recombinant DNA technology.

The polypeptides of the invention can exist in different forms, such as free acids, free bases, esters and other prodrugs, salts and tautomers, for example, and the invention includes all variant forms of the polypeptides. Thus, the invention encompasses the salt or pro-drug of a polypeptide.

The polypeptide of the invention may be administered in the form of a pharmaceutically acceptable salt. The invention thus includes pharmaceutically-acceptable salts of the polypeptide of the invention wherein the parent compound is modified by making acid or base salts thereof, for example the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glutamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

Salts of carboxyl groups of a polypeptide or polypeptide variant of the invention may be prepared in the usual manner by contacting the polypeptide with one or more equivalents of a desired base such as, for example, a metallic hydroxide base, e.g. sodium hydroxide; a metal carbonate or bicarbonate such as, for example, sodium carbonate or bicarbonate; or an amine base such as, for example, triethylamine, triethanolamine and the like.

### Products

The present invention further provides a product comprising a polypeptide of the invention and one or more additional antifungal agents (e.g. a second antifungal agent).

Suitably, the product of the present invention may comprise a second antifungal agent and, optionally, one or more additional antifungal agents (e.g. a third antifungal agent).

One or more additional antifungal agent(s) (e.g. a second antifungal agent) may be selected from the group of synthetic agents including polyenes, azoles, allylamines and echinocandins. Alternatively, the one or more additional antifungal agent(s) (e.g. the second antifungal agent) may be natural product including, by way of example, allium derivatives, essential oils and derivatives thereof, terpenoids, saponins, phenolic compounds, alkaloids. An additional antifungal agent (such as a second antifungal agent) may also include antifungal peptides or polypeptides and proteins.

The products of the present invention are effective in the treatment and prevention of Malassezia spp. infections. The agents of the product of the present invention may combine synergistically to provide surprisingly high antifungal activity. The amount of the second antifungal agent required is thus minimised. Alternatively, the agents of the product of the present invention may combine additively.

### Table 1: List of Second Antifungal Agents

Polyenes: Amphotericin B (including liposomal Amphotericin B and Amphotericin B lipid complex, Amphotericin B colloidal dispersion, Amphotericin B oral suspension), Candicidin, Filipin, Hamycin, Natamycin, Nystatin (including liposomal Nystatin), Rimocidin.

### Azoles: Imidazoles: Bifonazole, Butoconazole, Clotrimazole, Econazole, Enilconazole, Fenticonazole, Isoconazole, Ketoconazole, Miconazole, Omoconazole, Oxiconazole, Sertaconazole, Sulconazole, Tioconazole

### Triazoles: Albaconazole, Fluconazole, Isavuconazole, Itraconazole, Posaconazole, Ravuconazole, Terconazole, Voriconazole

### Thiazoles: Abafungin

### Allylamines: Amorolfine, Butenafine, Naftifine, Terbinafine

Echinocandins: Anidulafungin, Caspofungin, Micafungin, V-echinocandin (LY303366), Echinocandin B, Aculeacin, Aerothricins, Mulundocandin, Sporiofungins, Pneumocandins, Cryptocandin, WF11899 and related sulfate-derivatives, Arborcandins, Clavariopsins, Papulacandins, Corynecandin, Mer-WF3010, Fusacandin
Natural Products: Allium derivatives (e.g. allicin)
Essential oils and derivatives: Citronella oil, Chrysanthemum derivatives (e.g. β-basabolene, camphor and derivatives, α-curcumene, δ-elemene, farnesene, lyratyl acetate, α-pinene, β-pinene, piperitone, piperitonene, selena-4,7(11)-diene), Coconut oil (e.g. caprylic acid), Cypress derivatives (bornyl acetate, α-cadinol, muurolol), Lavender oil (including carvacrol, fenchone, linalool, limonene, myrtenol), Lemon myrtle oil, Neem seed oil, Olive Leaf Extract (e.g. oleuropein), Orange oil, Palmarosa oil, Patchouli oil, Tea tree oil.
Terpenoids: Diterpenoids (e.g. humiranthone, 16α-hydroxy-cleroda-3,13-(14)-2-diene-15,16-olide, patagonal), Sesquiterpenes and Sesquiterpene Lactones (e.g. atticin and 4-epi-sonchucarpolide), Triterpenes (e.g. celastrol, methyl angolensate, oleanolic acid, pristimerin, 1,3,7-trideacetylkhivorin, ursolic acid), Efumafungin, Arundifungin, Ascoteroside, Ergokonin A
Saponins: triterpene and steroid saponins
Phenolic compounds: Anthroquinones (e.g. alizarin, emodin, physcion, rhein), Arthrichtin, Coumarins and derivatives (e.g. daphnetin, esculin, esculetin, fraxetin, scopoletin, surangin B), Crassinervic acid, Flavones, Flavone glycosides, Flavonoids (e.g. biochanin A, dihydrobiochanin A, hyperoside, luteolin, 4-methoxy-5,7-dihydroxyflavone-6-C-glucosidetrifolin, Phellinsin A, Pinosylvin, Prenylated flavonoids, Stilbene derivatives

### Alkaloids: Anhydroevaxine, Berberine, Flinderisine, Haloxyline A, Haloxyline B, Haplamine, Jatorrhizine

Peptides & proteins: Peptides, including AcAFP, AFP-J, agrocibin, allicepin, angularin, brassiparin, brevinins, campesin, chromofungin, chromogranins, cicadin, cicerarin, coccinin, cordymin, curcurmoschin, defensins, drosomycin, eryngin, gallerimycin, globopeptin, gymnin, halocidins, hevein-type peptides, histatins, hypogin, isarfelin, iturins, knottin-type peptides (e.g. psacotheacin), metchnikowin, mycobacillin, mytimicin, PAF-26, pleurostrin, Pm-AMP1, pomegranin, scarabaecin, SP-B, stendomycin, vulgarinin, Vv-AMP1.
Enzymes, including chitinase, lysozyme; Proteins: including chitin-binding proteins, thaumatin-like proteins.
Others: Antimycin A, Aureobasidins, Australifungin, Benanomycins, Benzoic acid, Chitosan, Ciclopirox, Clioquinol, Flucytosine, Fumonisin B1, Griseofulvin, Halprogin/Haloprogin, Hypoxysordarin, Iodine (including potassium iodide), Khafrefungin, Lipoxamycin, Minimoidin, Nikkomycins, Piroctone olamine, Polygodial, Polyoxins, Povidone-Iodine, Pramicidins, Pyrithiones, Rustmicin, Selenium (including selenium sulphide), Silver (including colloidal silver), Sordarin, Sphingofungins, Tar, Tolnaftate, Undecylenic acid, Valinomycin, Viridiofungins, Xylarin, Zinc, Zinc pyrithione, Zofimarin.

In one embodiment, one additional antifungal agent (e.g. the second antifungal agent) is a coumarin compound, for example a glycosidic coumarin compound.

The term “coumarin” as used herein includes reference to a compound comprising a chromenone ring. In one class of coumarin compounds, the chromenone ring is a chromen-2-one ring, while in another class the chromenone ring is a chromen-4-one ring. Many of the known coumarins are of the former class. Examples of coumarins of the latter class include quercetins and derivatives thereof.

The terms “glycosidic compound” as used herein are interchangeable and includes reference to any of the class of compounds that yield a sugar and an aglycone upon hydrolysis.

Examples of coumarin compounds include 6-Bromo-3-butyrylcoumarin, 6-Bromocoumarin-3-carboxylic acid, 6-Bromocoumarin-3-carboxylic acid, 6,8-Dibromocoumarin-3-carboxylic acid, 3-Chlorocoumarin, 4-Chloro-3-nitrocoumarin, 7-Amino-4-(trifluoromethyl)coumarin, 7-Amino-4-(trifluoromethyl)coumarin, 7-Hydroxy-4-(trifluoromethyl)coumarin, 2,3,6,7-Tetrahydro-9-trifluoromethyl-1H,5H-quinolizino(9,1-gh)coumarin (Coumarin 153), 6-Bromo-3-(2,3-dichlorophenylcarbamoyl)-coumarin, 7-Ethoxy-4-(trifluoromethyl)coumarin, 7-Hydroxy-4-(trifluoromethyl)coumarin, 7-Methoxy-4-(trifluoromethyl)coumarin, 7-(Phenylacetamido)-4-(trifluoromethyl)coumarin, 3-Acetyl-6-bromocoumarin, L-Alanine-7-amido-4-methylcoumarin trifluoroacetate, 6-bromocoumarin, 6-bromo-3-cyanocoumarin, 6-bromo-3-cyano-4-methylcoumarin, 6-bromo-4-hydroxycoumarin, 6-bromomethyl-7-acetoxycoumarin, 4-(bromomethyl)-6,7-dimethoxycoumarin, 4-(bromomethyl)-7-methoxycoumarin, 6-bromo-4-methyl-3-phenylcoumarin, 3-butyryl-6,8-dibromocoumarin, 6-chlorocoumarin, 6-chloro-3-cyanocoumarin, 6-chloro-3-cyano-4,7-dimethylcoumarin, 6-chloro-3-cyano-4-methylcoumarin, 6-chloro-3-cyano-4,7-dimethyl-3-phenylcoumarin, 6-chloro-4-hydroxycoumarin, 6-chloro-7-hydroxy-4-(methoxymethyl)coumarin, 6-chloro-4-hydroxy-7-methylcoumarin, 6-chloro-4-hydroxy-4-(trifluoromethyl)coumarin, 6-chloro-4-methyl-7-phenylcoumarin, 4-chloro-3-nitrocoumarin, 6-(3-chloropropoxy)-4-methylcoumarin, 3-cyano-6,8-dibromo-4-methylcoumarin, 3-cyano-6,8-dichloro-4-methylcoumarin, 3-cyano-6,7-dichloro-4-methylcoumarin, 3-cyano-6-fluoro-4-methylcoumarin, 6,8-dibromo-4-hydroxycoumarin, 6,8-dibromocoumarin-3-carboxylic acid, 6,8-dibromo-4-methyl-3-phenylcoumarin, 6,7-dichloro-4-hydroxycoumarin, 6,8-dichloro-4-hydroxycoumarin, 6,7-dichloro-4-methyl-3-phenylcoumarin, 6,8-dichloro-4-methyl-3-phenylcoumarin, ethyl 6,8-dibromocoumarin carboxylate, 6-fluoro-4-hydroxycoumarin, 6-fluoro-4-methyl-3-phenylcoumarin, 7-hydroxy-4-(trifluoromethylphenyl)coumarin.

Examples of glycosidic coumarin compounds include Esculin (6,7-esculin or 2,6-esculin), fraxin, 4-methylumbelliferyl β-D-glucopyranoside, 4-methylumbelliferyl α-D-galactopyranoside, Esculetin-7-O-glucoside (cichoriin), 4-methylumbelliferyl α-D-mannopyranoside, 4-methylumbelliferyl α-L-fucopyranoside, 4-methylumbelliferyl-α-L-arabinopyranoside, 4-methylumbelliferyl β-D-glucopyranoside, 4-methylumbelliferyl β-D-galactopyranoside, 4-methylumbelliferyl β-D-glucuronide, 4-methylumbelliferyl N-acetyl-β-D-glucosaminide, 4-methylumbelliferyl N-acetyl-β-D-galactosaminide, 4-methylumbelliferyl β-D-xylopyranoside, 4-methylumbelliferyl β-D-lactopyranoside, 4-trifluoromethylumbelliferyl β-D-glucopyranoside, 4-trifluoromethylumbelliferyl β-D-galactopyranoside, 6,8-difluoro-4-methylumbelliferyl β-D-glucopyranoside, quercetin 3-β-D-glucoside, quercetin 3-rhamnoside, quercetin 3-D-xyloside.

Suitably, one additional antifungal agent may be esculin.

According to one embodiment, one additional antifungal agent (e.g. the second antifungal agent) is a non-peptide.

According to a further embodiment, one additional antifungal agent (e.g. the second antifungal agent) is an echinocandin. For example the echinocandin may be selected from the group consisting of Echinocandin B, Aculeacin, Aerothricins, Mulundocandin, Sporiofungins, Pneumocandins, Cryptocandin, WF11899 and related sulfate-derivatives, Arborcandins, Clavariopsins, Papulacandins, Corynecandin, Mer-WF3010, Fusacandin.

In an alternative embodiment, one additional antifungal agent (e.g. the second antifungal agent) is zinc pyrithione.

### Administration and Pharmaceutical Formulations

A further aspect of the invention provides a pharmaceutical composition comprising a pharmaceutically effective amount of a polypeptide or product of the invention.

The ratio of the polypeptide of the invention to the second agent in the products of the invention may be from 1:10 to 10:1; generally at least approximately 1:1 or at least 2:1 for example at least 3:1 or 4:1. Alternatively, the ratio of the antibiotic agent to the second agent in the products of the invention may be from 1:1 to 100:1.

The active agents may be administered simultaneously, sequentially or separately. The active agents may be provided as a combination package. The combination package may contain the product of the invention together with instructions for simultaneous, separate or sequential administration of each of the active agents. For sequential administration, the active agents can be administered in any order.

The composition also includes a pharmaceutically and/or cosmetically acceptable carrier, excipient or diluent. The phrases “pharmaceutically acceptable” and “cosmetically acceptable” are employed herein to refer to those compounds, materials, compositions, and/or dosage forms which are, within the scope of sound medical judgment, suitable for use in contact with the tissues of human beings or, as the case may be, an animal without excessive toxicity, irritation, allergic response, or other problem or complication, commensurate with a reasonable benefit/risk ratio.

To prepare the composition, polypeptides are synthesised or otherwise obtained, purified as necessary or desired, and then lyophilised and stabilised. The polypeptide can then be adjusted to the appropriate concentration and optionally combined with other agents.

Thus, one or more suitable unit dosage forms comprising the therapeutic polypeptides of the invention can be administered by a variety of routes including oral, dermal, topical, parenteral (including subcutaneous, intravenous, intramuscular and intraperitoneal), vaginal, rectal, dermal, transdermal, intrathoracic, intrapulmonary and intranasal (respiratory) routes.

Preferably, the polypeptides of the invention are for topical administration for example to the skin, hair or nails, especially the face or scalp.

For topical administration, the active agents may be formulated as is known in the art for direct application to a target area, for example the scalp, hair and skin. Forms chiefly conditioned for topical application take the form, for example, of shampoos, conditioners, other hair products, lotions, laquers, creams, milks, gels, powders, dispersions or microemulsions, lotions thickened to a greater or lesser extent, impregnated pads, ointments or sticks, aerosol formulations (e.g. sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic polypeptides of the invention may be for dermal administration for example via patches or bandages.

Preferably, the active agents are formulated for application to the scalp, for example, in the form of a shampoo, conditioner, serum, gel or spray.

These formulations can contain pharmaceutically and/or cosmetically acceptable carriers, vehicles and adjuvants that are well-known in the art. It is possible, for example, to prepare solutions using one or more organic solvent(s) that is/are acceptable from the physiological standpoint, chosen, in addition to water, from solvents such as acetone, acetic acid, ethanol, isopropyl alcohol, dimethyl sulphoxide, glycol ethers such as the products sold under the name “Dowanol”, polyglycols and polyethylene glycols, C₁-C₄ alkyl esters of short-chain acids, ethyl or isopropyl lactate, fatty acid triglycerides such as the products marketed under the name “Miglyol”, isopropyl mytrisate, animal, mineral and vegetable oils and polysiloxanes.

### Use

The polypeptides or products of the invention may be useful in the treatment or prevention of fungal infections caused by Malassezia spp and/or a Malassezia spp associated condition. For example, the polypeptides or products of the invention may be useful in the treatment or prevention of: dermatitis (e.g. seborrheic dermatitis or atopic dermatitis), dandruff, pityriasis/tinea versicolor, pityriasis/tinea folliculitis, Malassezia folliculitis, acne vulgaris, dacryocystitis, seborrhoeic blepharitis, otitis externae, confluent and reticulated papillomatosis, nodular hair infection, psoriasis, mastitis, sinusitis, septic arthritis, peritonitis, neontala pustulosis and catheter-related fungemia.

The infection may be caused by or the condition associated with any Malassezia spp (formerly known as Pityrosporum spp.), e.g. Malassezia furfur, Malassezia pachydermatis, Malassezia globosa, Malassezia obtusa, Malassezia restricta, Malassezia slooffiae, Malassezia sympodialis, M. dermatis, M. japonica, M. nana and M. yamatoensis. Typically, the infection is caused by or the condition associated with Malassezia furfur, Malassezia globosa, Malassezia pachydermatis Malassezia restricta or Malassezia sympodialis.

Thus, a further aspect of the invention provides the use of a polypeptide or product according to the invention, or a pharmaceutically and/or cosmetically acceptable salt thereof, in the manufacture of a medicament for the treatment or alleviation of an infection contributed to or caused by Malassezia spp.

The invention further provides the use of a polypeptide or product of the invention, or a pharmaceutically acceptable salt thereof, in the manufacture of a medicament for the treatment or alleviation of a disease or condition contributed to or caused by a Malassezia spp. infection.

A disease or condition contributed to or caused by a Malassezia spp. infection may include infections of the skin including pityriasis versicolor, seborrhoeic dermatitis (including dandruff [pityriasis capitis], sebopsoriasis and facial or scalp psoriasis), secondary infections related to acne vulgaris, folliculitis, neonatal pustulosis, blepharitis, papillomatosis (confluent and reticulated), facial atopic dermatitis, invasive pityrosporosis (immunodeficient individuals) and white piedra. Given the lipophilic nature of most species of Malassezia spp., fungaemia, catheter-related infections and sepsis due to Malassezia furfur may occur particularly in patients who are on parenteral nutrition with lipids. Colonization of catheters with Malassezia spp. may occur in absence of lipid administration as well.

In one embodiment, the patient is a mammal, in particular human.

In another embodiment, the patient is an animal. In this regard, the animal may be any animal which is susceptible to a Malassezia spp. infection.

Suitably, the animal may be a domesticated animal such as a dog or a cat.

The extent of protection includes counterfeit or fraudulent products which contain or purport to contain a compound of the invention irrespective of whether they do in fact contain such a compound and irrespective of whether any such compound is contained in a therapeutically effective amount.

Features, integers, characteristics, compounds, chemical moieties or groups described in conjunction with a particular aspect, embodiment or example of the invention are to be understood to be applicable to any other aspect, embodiment or example described herein unless incompatible therewith.

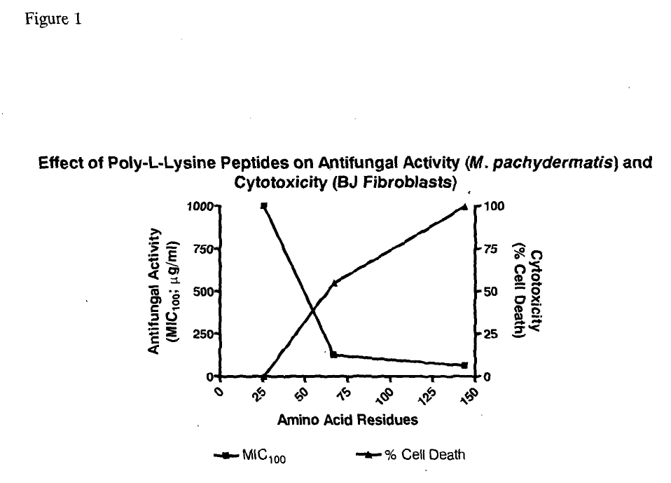

FIG. 1 shows the “Effect of Poly-L-Lysine Polypeptides on Antifungal Activity (M. pachydermatis) and Cytotoxicity (BJ Fibroblasts)”.

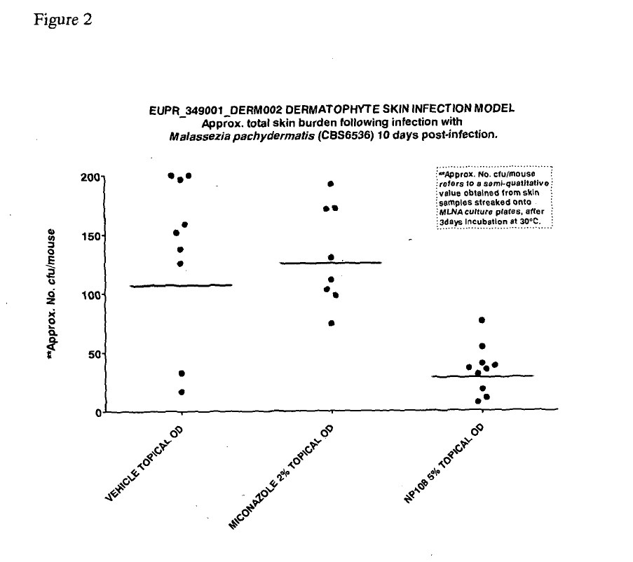

FIG. 2 shows the approximate total skin burden of mice following dermal infection with Malassezia pachydermatis 10 days post-infection and following treatment with NP108 or Miconazole.

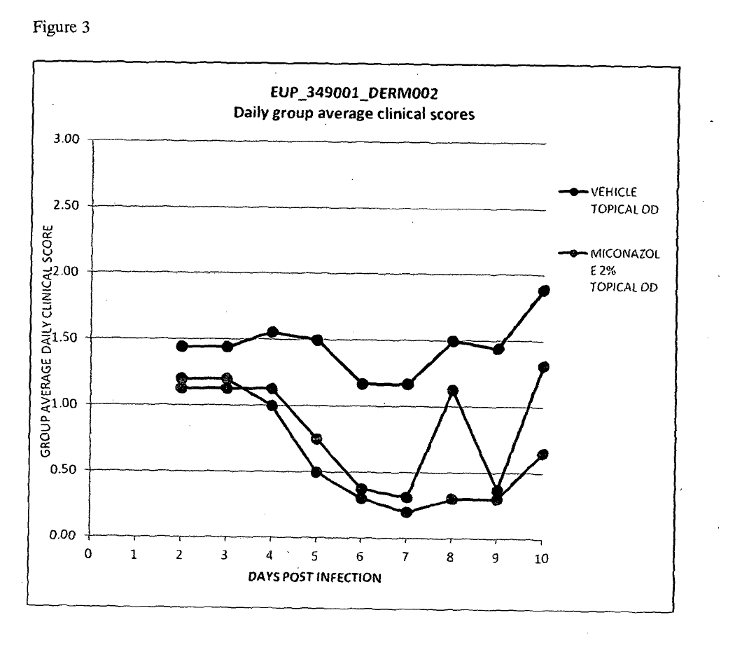

FIG. 3 shows the daily group average clinical scores of mice following dermal infection with Malassezia pachydermatis 10 days post-infection and following treatment with NP108, or Miconazole.

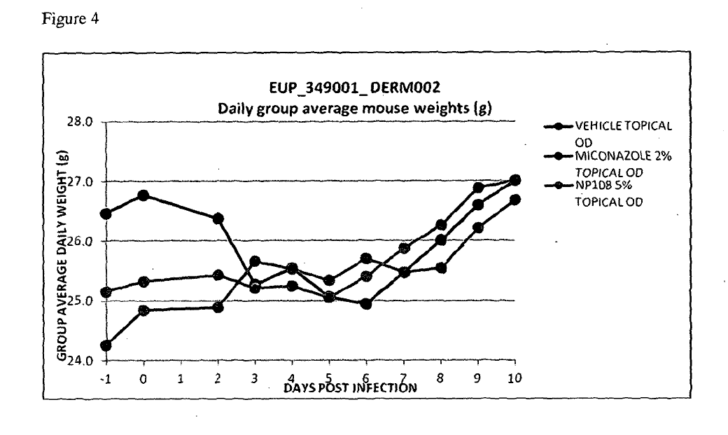

FIG. 4 shows the daily group average weights (g) of mice following dermal infection with Malassezia pachydermatis 10 days post-infection and following treatment with NP108 or Miconazole.

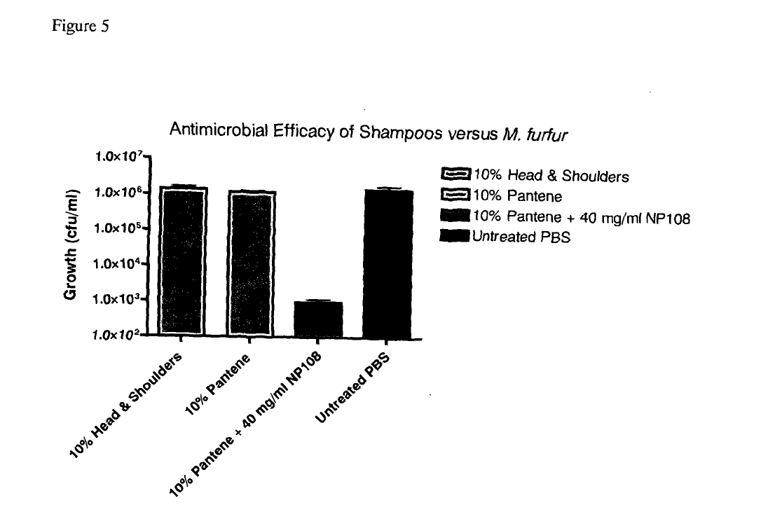

FIG. 5 shows the antimicrobial efficacy of shampoos versus Malassezia furfur

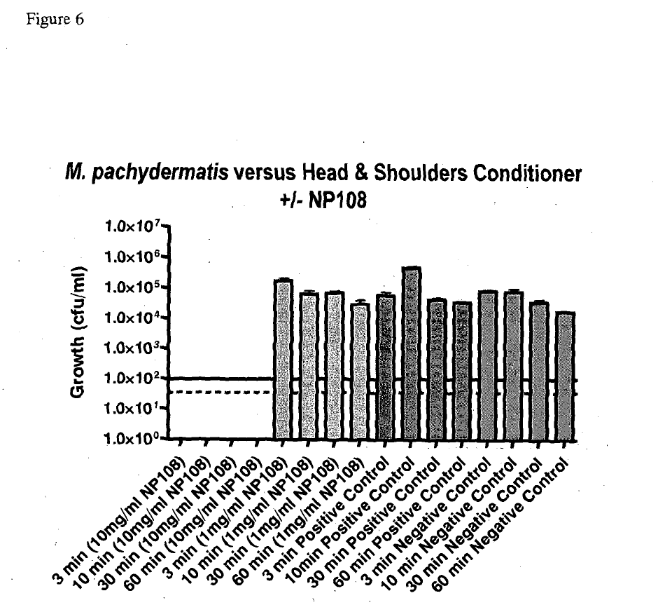

FIG. 6 shows the antimicrobial efficacy of Head & Shoulders Conditioners versus Malassezia pachydermatis

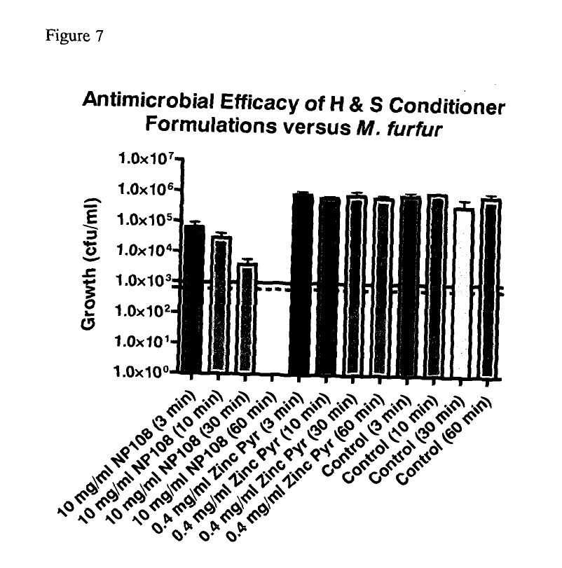

FIG. 7 shows the antimicrobial efficacy of Head & Shoulders Conditioner formulations versus Malassezia furfur

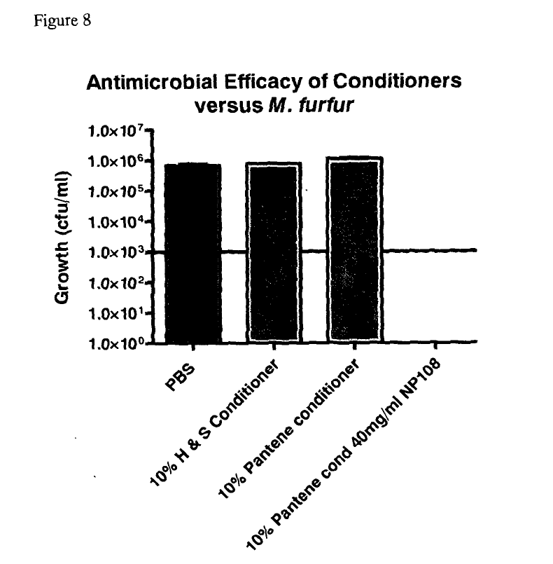

FIG. 8 shows the antimicrobial efficacy of Conditioners versus Malassezia pachydermatis

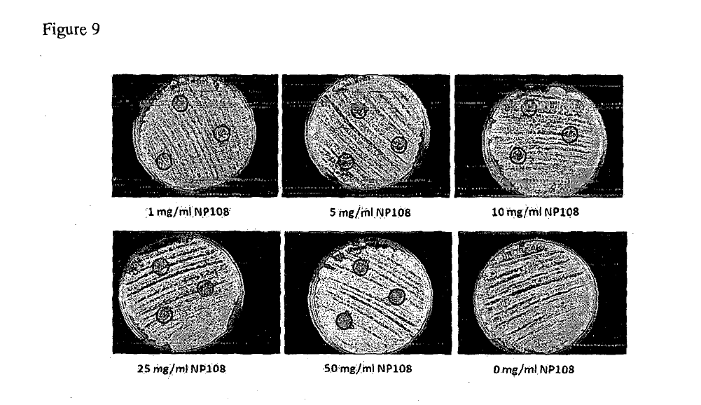

FIG. 9: shows the antimicrobial activity of NP108 in a 65% (w/v) PEG14,000 gel suitable for application to the skin and hair/fur of humans and animals versus M. furfur DSMZ6170 grown on solid media.

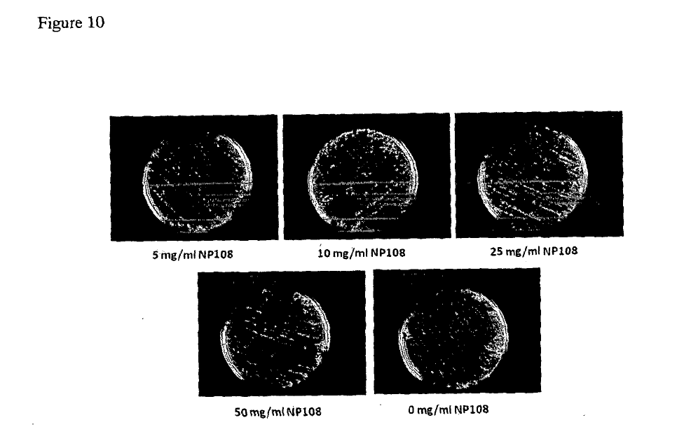

FIG. 10: shows the antimicrobial activity of NP108 in a 65% (w/v) PEG14,000 gel suitable for application to the skin of and hair/fur humans and animals versus M. pachydermatis CBS6536 grown on solid media

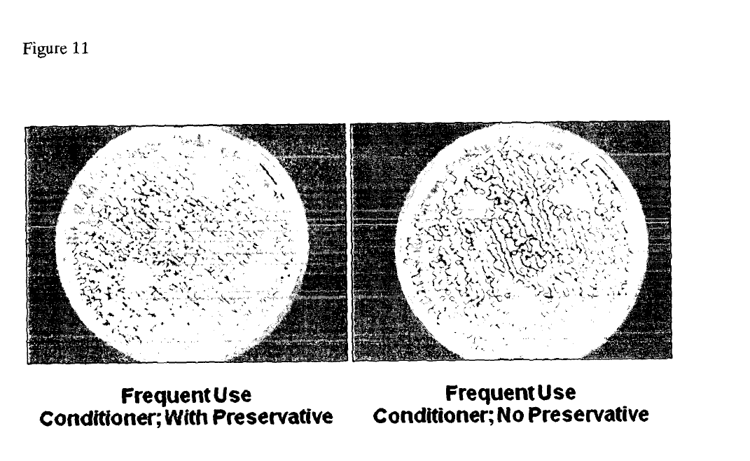

FIG. 11: shows the effect of 0.5% (w/v) NP108+0.5% (w/v) Esculin on the growth of M. furfur DSMZ6170 in a Frequent Use Conditioner with and without 0.2% (w/v) Optiphen MIT Plus preservative.

The following Example illustrates the invention.

### EXAMPLE

### Materials and Methods

### Polypeptide Synthesis

All polylysine polypeptides were produced either by solid-phase synthesis under contract by a polypeptide supplier, PolyPeptide Laboratories France SAS (Strasbourg, France), or purchased from Sigma-Aldrich Chemical Company Ltd. (Poole, UK). Characteristics of the polypeptides, including molecular weights in terms of mass (Da) and number of amino acid residues can be found in Table 1.

### Determination of the Minimum Inhibitory Concentration of Materials Versus Malassezia Spp.

The minimum inhibitory concentration (MIC) of all materials was determined according to methods described in the Clinical and laboratory Standards institute Approved Standard “Reference Method for Broth Dilution Antifungal Susceptibility Testing of Yeasts—Third Edition (M27-A3)” with the following modifications. In place of RPMI-1640 liquid medium, Modified Christensen's Medium, without the addition of agar, was used. The MIC for M. pachydermatis CBS6536 was determined against 1×10⁶-5×10⁶ cells/ml, rather than the normal 5×10²-2.5×10³ cells/ml, to improve reproducibility and consistency of results (Tables 1 & 2).

### Toxicity Analysis

Haematotoxicity versus 10% human red blood cells and cyctotoxicity versus human dermal fibroblast (BJ) cells and human lung epithelial (A549) cells was determined by standard in vitro procedures known to those skilled in the art (Table 1 and FIG. 1).

Determination of the Efficacy of NP108 Against Malassezia pachydermatis (CBS6536) in a Murine Model of Localised Dermatophyte Skin Infection (10 Days Post-Infection)

In this Malassezia spp. skin infection study, 30 male CD1 mice (10 per treatment group) were scored daily for 10 d post-infection based on the clinical observations of the infected area. Mice were treated with test article, 5% (w/v) NP108, 2% (w/v) miconazole or vehicle commencing 48 h post-infection for 6 d. Groups of 10 mice were euthanized for culture of skin tissue 10 d post-infection. Skin samples were dissected into 10 pieces per mouse and each piece streaked onto Modified Leeming Notman agar (MLNA) for culture at 30° C. for up to 7 d. Data from these samples has been recorded as number positive for Malassezia pachydermatis CBS6536 growth per mouse and approximate number of quantifiable cfu per streaked sample; plates were read after 3 d and 7 d incubation. Test article NP108 was supplied as a 5% (w/v) dosing preparation in 65% PEG14000, supplied ready to administer without any reconstitution or dilution required, as was vehicle preparation also. Treatment with test article was well tolerated throughout the study. In general some mice had reduced food intake resulting in some reduction in weight but this was in proportion to the level of immunosuppression and severity of infection of the animals.

A high level of Malassezia pachydermatis CBS6536 skin infection was established in this model, with 2 vehicle mice reaching the highest clinical score (Score 3.0=significant crusting/erythema) by the end of the study. The vehicle treatment group scored higher than both NP108 and miconazole throughout the duration of the study (Group ave. Day+10 clinical score 1.89, 96.7% positive skin cultures).

Test article 5% (w/v) NP108 demonstrated significant efficacy in reducing both clinical observations of infected areas (Group ave. Day+10 clinical score 0.65 (no visible lesions to slight change in skin colour/texture), P<0.0001, StatsDirect—Kruskal-Wallis: all pairwise comparisons (Conover-Inman) and approximate number of cfu obtained from skin biopsies (3 days incubation, Group ave. 34.6 cfu/mouse, P=0.0004, StatsDirect—Kruskal-Wallis, 84% positive skin cultures, P=0.0008, StatsDirect—Fisher's Exact Test (Group Ave. Data)) compared with vehicle only treated mice (Group ave. Day+10 clinical score 1.89 (slight change in skin colour/texture to redness and slight crusting), Group ave. 135.8 cfu/mouse, 96.7% positive skin cultures) (FIGS. 2-4).

Efficacy of the comparator drug 2% (w/v) miconazole was inferior to the test article, 5% (w/v) NP108, for all parameters measured in this study (Group ave. Day+10 clinical score 1.31 (slight change in skin colour/texture to redness and slight crusting), Group ave. 131.4 cfu/mouse, 100% positive skin cultures) and did not demonstrate a statistically significant improvement over vehicle only treated mice (P>0.05 NS, StatsDirect—Kruskal-Wallis: all pairwise comparisons (Conover-Inman)).

In conclusion, test article 5% (w/v) NP108 applied topically once a day for 6 d has shown some significant efficacy against Malassezia pachydermatis (CBS6536), in improving the severity of clinical observations and reducing the dermatophyte burdens from skin biopsies. Topical 5% (w/v) NP108 was effective at reducing the burden of Malassezia pachydermatis in a murine model of cutaneous infection. The efficacy of 5% (w/v) NP108 was superior to a marketed cream containing 2% (w/v) Miconazole (Daktarin).

### Antifungal Efficacy of NP108 in a Vehicle Suitable for Topical Delivery

NP108 was prepared aseptically in a sterile PEG14000 vehicle (65% (w/v) PEG14,000, X % (w/v) NP108, Y % (w/v) deionised H₂O; to 100% (w/v)) for testing purposes. NP108 was added to the vehicle at the following concentrations (% (w/v); 0.1, 0.5, 1.0, 2.5 and 5.0. As positive controls the PEG14000 vehicle was prepared containing 1% (w/v) ketoconazole and 1% (w/v) clotrimazole and negative controls were prepared without antifungal agents or NP108 with the balance made up with sterile deionised H₂O (sdH₂O). All yeast inocula for this experiment were prepared to the 0.5 McFarland Standard. All experiments were carried out in triplicate.

Plates of Sabouraud Dextrose medium (SDA) made with 1.5% (w/v) agarose, rather than agar. Plates were inoculated with Malassezia spp. (M. furfur DSMZ6170 or M. pachydermatis CBS6536) and within 15 min of inoculation, 5 mg of antifungal agents in the PEG14000 vehicle were applied to the plates. Plates were incubated aerobically at 30° C. for 48-72 h. Zones of clearance were recorded photographically and measured using a ruler (FIGS. 9 & 10).

### Antifungal Efficacy of NP108 in a Shampoo Vehicle

The antifungal efficacy of NP108 was tested in a shampoo vehicle. The following materials were tested:
1. 10% (v/v) Head & Shoulders shampoo (anti-dandruff shampoo)
2. 10% (v/v) Pantene shampoo (“normal” shampoo)
3. 10% (v/v) Pantene shampoo (“normal” shampoo)+4% (w/v) NP108
4. Phosphate Buffered Saline (PBS)

All yeast inocula for this experiment were prepared to the 0.5 McFarland Standard. 400 μl of yeast inoculum was exposed to 100 μl of the materials described above for 1 h at 37° C., followed by washing to remove all traces of the materials. Serial dilutions of the yeast inocula were prepared (10⁰-10⁻⁵; 10-fold dilutions) and 100 μl was spread on Modified Christensen's Medium and incubated at 30° C. for 48 h and numbers of surviving colonies counted. All experiments were carried out in triplicate.

In order to determine whether the antifungal effects of NP108 could be observed in a suitable vehicle, known concentrations were added to selected shampoo's.

In the following experiment, all samples of M. furfur DSMZ6170 (0.5 McFarland Standard) were exposed to 10% (v/v) Head & Shoulders (H & S) Shampoo, 10% (v/v) Pantene Shampoo and 10% (v/v) Pantene Shampoo containing 40.0 (4.0%) mg/ml NP108. The negative control samples were exposed to phosphate-buffered saline (PBS) alone. H & S Shampoo contains zinc pyrithione (˜1%) as an antifungal agent proven to kill Malassezia spp. Pantene shampoo has an almost identical formulation to H & S Shampoo, but does not contain zinc pyrithione.

As can be seen in FIG. 5 the shampoos alone do not kill M. furfur DSMZ6170 at this concentration, whereas the shampoo supplemented with 40 mg/ml NP108 (1.0%) kills M. furfur DSMZ6170 after 60 min exposure. These experiments were carried out in triplicate. The results are mean cfu/ml and the error bars are the standard error of the mean.

### Antifungal Efficacy of NP108 in a Conditioner Vehicle

The antifungal efficacy of NP108 was tested in a conditioner vehicle. The following materials were tested:
1. 10% (v/v) Head & Shoulders conditioner (anti-dandruff conditioner)
2. 10% (v/v) Pantene conditioner (“normal” conditioner)
3. 10% (v/v) Pantene conditioner (“normal” conditioner)+4% (w/v) NP 108
4. Phosphate Buffered Saline (PBS)

All yeast inocula for this experiment were prepared to the 0.5 McFarland Standard. 400 μl of yeast inoculum was exposed to 100 μl of the materials described above for 1 h at 37° C., followed by washing to remove all traces of the materials. Serial dilutions of the yeasts were prepared (10⁰-10⁻⁵; 10-fold dilutions) and 100 μl was spread on Modified Christensen's Medium and incubated at 30° C. for 48 h and numbers of surviving colonies counted. All experiments were carried out in triplicate.

In a further experiment, the above experiment was repeated using 10% (v/v) Head & Shoulders Conditioner alone, containing the following concentrations of NP108 (% (w/v); 0, 0.125, 0.25, 0.5, 1.0, 2.0 and 4.0.

In a further experiment, the effect of incubation time on killing of Malassezia spp. was investigated. The following materials were tested:
1. 0.04% (w/v) Zinc Pyrithione in 19.2% (v/v) Head &Shoulders conditioner
2. 1.0% (w/v) NP108 in 19.2% (v/v) Head & Shoulders conditioner
3. 19.2% (v/v) Head & Shoulders conditioner

All yeast inocula for this experiment were prepared to the 0.5 McFarland Standard. 400 d of yeast inoculum was exposed to 100 μl of the materials described above and incubated at 30° C. for 0, 3, 10, 30 or 60 min, followed by washing to remove all traces of the materials. Serial dilutions of the yeasts were prepared (10⁰-10⁻⁵; 10-fold dilutions) and 100 μl was spread on Modified Christensen's Medium and incubated at 30° C. for 48 h and numbers of surviving colonies counted. All experiments were carried out in triplicate.

In order to determine whether the antifungal effects of NP108 could be observed in a suitable vehicle, known concentrations were added to selected shampoo's and conditioner's.

In the following experiment, all samples of M. pachydermatis CBS6536 (0.5 McFarland Standard) were exposed to 10% (v/v) Head & Shoulders (H & S) Conditioner, containing 0 (0%), 1.0 (0.1%) or 10.0 (1.0%) mg/ml NP108. The positive control samples were exposed to 10% (v/v) Head & Shoulders Conditioner. The negative control samples were exposed to water alone.

As can be clearly seen (FIG. 6), 10 mg/ml NP108 (1.0%) kills M. pachydermatis CBS6536 after as little as 3 min exposure in H & S conditioner, whereas 1 mg/ml NP108 (0.1%) had no antifungal activity. H & S Conditioner (Positive Control) and Water (Negative Control) had no antifungal activity.

In the following experiment, all samples of M. furfur DSMZ6170 (0.5 McFarland Standard) were exposed to 10% (v/v) H & S Conditioner, containing 10.0 (1.0%) mg/ml NP108 or 0.4 mg/ml zinc pyrithione (the active ingredient in H & S shampoo). The control samples were exposed to 10% (v/v) H & S Conditioner. The negative control samples were exposed to water alone (data not shown).

As can be clearly seen (FIG. 7), 10 mg/ml NP108 (1.0%) demonstrates antifungal activity versus M. furfur DSMZ6170 after as little as 3 min exposure in H & S conditioner and demonstrates, a time-dependent increase in antifungal activity. Complete killing of M. furfur DSMZ6170 is achieved after 60 min exposure. H & S Conditioner (Control) and 0.4 mg/ml zinc pyrithione had no antifungal activity. 0.4 mg/ml zinc pyrithione is sufficient to kill M. furfur DSMZ6170 under in vitro conditions.

In the following experiment, all samples of M. furfur DSMZ6170 (0.5 McFarland Standard) were exposed to 10% (v/v) H & S Conditioner or 10% (v/v) Pantene Conditioner and a further treatment of 40 mg/ml NP108 (4.0%) in 10% (v/v) Pantene Conditioner. The control sample was exposed to PBS alone. All incubations were carried out for 60 min.

As can be clearly seen (FIG. 8), 40 mg/ml NP108 (4.0%) demonstrates antifungal activity versus M. furfur DSMZ6170 after 60 min exposure in Pantene conditioner completely killing M. furfur DSMZ6170. The conditioners demonstrated no antifungal activity.

In a further experiment, samples of M. furfur DSMZ6170 (0.5 McFarland Standard) were exposed to an alternative conditioner (Proprietary Frequent Use Conditioner+/−0.2% (w/v) Optiphen MIT Plus preservative containing 0.5% (w/v) NP108 and 0.5% (w/v) esculin (a coumarin glycoside) (FIG. 11). The control sample was exposed to conditioner alone which had no antifungal activity (data not shown). All incubations were carried out for 60 min.

As can be clearly seen (FIG. 11), 5 mg/ml NP108 (0.5%) and 5 mg/ml (0.5%) esculin demonstrates antifungal activity versus M. furfur DSMZ6170 after 60 min exposure in the proprietary frequent use conditioner, killing M. furfur DSMZ6170 in clear zones around the area of application. The conditioner alone demonstrated no antifungal activity (data not shown).

TABLE 1
Antimicrobial Efficacy of Poly-L-Lysine versus M. pachydermatis CBS6536

| | | | | | | | | Cytotoxicity |
|---|---|---|---|---|---|---|---|---|
| | Mol Wt | Mol Wt | MIC₅₀ | MIC₈₀ | MIC₁₀₀ | Haemolysis | Cytotoxicity | (A549; LD₅₀; |
| Polypeptide | (Da) | (aa) | (μg/ml) | (μg/ml) | (μg/ml) | (LD₅₀ μg/ml) | (BJ; LD₅₀ (μg/ml)) | mg/ml) |
| NP111 | <1795 | <12 | 500 | 500-1000 | 1000 | >10,000 | | |
| NP106 | 4,000 | ~26 | 250-500 | 250-500 | 1000 | >10,000 | >10,000 | >20,000 |
| NP114 | 6,000 | ~38 | 3.9-7.8 | 7.8-15.6 | 15.6 | ND | | |
| NP108 | 10,500 | ~67 | 62.5-125 | 62.5-125 | 125 | >10,000 | 4,500 | 15,000 |
| NP107 | 13,800 | ~88 | 31.25-62.5 | 62.5 | 125 | >10,000 | | |
| NP113 | 16,900 | ~108 | 31.25 | 31.25-62.5 | 62.5 | ND | | |
| NP115 | 20,000 | ~128 | 15.6 | 15.6-31.25 | 31.25 | ND | | |
| NP101 | 25,200 | ~161 | 15.6 | 31.25 | 62.5 | >10,000 | 2,700 | 210 |
| NP109 | 25,700 | ~165 | 31.25 | 31.25-62.5 | 62.5 | >10,000 | | |
| NP110 | >29,607 | >189 | 15.6-31.25 | 31.25-62.5 | 62.5 | >10,000 | | |

Summary:
Poly-L-lysine polypeptides of 38-189 aa residues demonstrate significant antifungal activity versus Malassezia spp.
Poly L-lysine polypeptides of >161 aa residues demonstrate significant cytotoxic activity versus BJ fibroblasts and A549 lung epithelial cells

TABLE 2
Antimicrobial efficacy of NP108 versus M. furfur and M. pachydermatis

| | MIC₅₀ (μg/ml) | | MIC₈₀ (μg/ml) | | MIC₁₀₀ (μg/ml) | |
|---|---|---|---|---|---|---|
| | Range | Median | Range | Median | Range | Median |
| M. furfur | 125-500 | 250 | 500-2000 | 1000 | 2000-4000 | 2000 |
| DSMZ6170 | | | | | | |
| M. pachydermatis | 15.63-31.25 | 31.25 | 31.25-125 | 62.5 | 31.25-125 | 62.5 |
| CBS6536 | | | | | | |
| M. pachydermatis | | 15.6 | | 15.6-31.3 | | 31.3 |
| NCPF3667 | | | | | | |

These results are from triplicate samples in a single experiment and the result of three independent experiments

1.-29. (canceled)

30. A method of treating or preventing a fungal infection caused by Malassezia spp. and/or a Malassezia spp. associated condition comprising administering a polypeptide to a subject, wherein the polypeptide comprises a sequence of about 25 to 200 amino acids wherein substantially all of the amino acids in said sequence are lysine.

31. A method as claimed in claim 30 wherein the polypeptide comprises a sequence of consecutive lysine residues.

32. A method as claimed in claim 30 wherein the polypeptide comprises a sequence of about 38 to 189 lysine residues.

33. A method as claimed in claim 32 wherein the polypeptide comprises a sequence of 50 to 150 lysine residues.

34. A polypeptide as claimed in claim 33 wherein the polypeptide comprises a sequence of 50 to 125 lysine residues.

35. A polypeptide as claimed in claim 30 wherein the polypeptide is polylysine.

36. A method as claimed in claim 30 wherein the polypeptide is administered topically.

37. A method as claimed in claim 36 wherein the polypeptide is administered to the face or scalp.

38. A method according to claim 30 wherein the subject is a human.

39. A method according to claim 30 wherein the subject is an animal.

40. A method according to claim 39 wherein the fungal infection caused by Malassezia spp. and/or the Malassezia spp. associated condition is otitis, dermatitis or mastitis.

41. A method according to claim 30 wherein the fungal infection or Malassezia spp. associated condition is selected from the group consisting of: dermatitis (e.g. seborrhoeic dermatitis or atopic dermatitis), dandruff, pityriasis/tinea versicolor, pityriasis/tinea folliculitis, Malassezia folliculitis, acne vulgaris, dacryocystitis, seborrhoeic blepharitis, otitis externae, confluent and reticulated papillomatosis, nodular hair infection, psoriasis, mastitis, sinusitis, septic arthritis, peritonitis, neontala pustulosis and catheter-related fungemia.
